(19) United States
(12) Patent Application Publication
(10) Pub. No.: US 2009/0275034 A1
(43) Pub. Date: Nov. 5, 2009

(54) TEMPERATURE CONTROL SYSTEM

(75) Inventors: Sepehr Kiani, Watertown, MA (US); Abhjeet Shinde, Medford, MA (US)

Correspondence Address: COOLEY GODWARD KRONISH LLP;ATTN: Patent Group, Suite 1100, 777 - 6th Street, NW, WASHINGTON, DC, 20001, US

(73) Assignee: HELICOS BIOSCIENCES CORPORATION, Cambridge, MA (US)

(21) Appl. No.: 12/397,937

(22) Filed: Mar. 4, 2009

Related U.S. Application Data
(60) Provisional application No. 61/034,131, filed on Mar. 5, 2008.

(51) Int. Cl. C12Q 1/68; C12M 1/02

(52) U.S. Cl. 435/6; 435/303.1

(57) ABSTRACT

Single molecule technologies generally require sensitive optical detection and the ability to operate at multiple temperatures simultaneously in different parts of the instrument. The system for controlling the temperature of a microfluidic device and methods for controlling the temperature of sequencing reactions includes a chamber for receiving a microfluidic device, a heating control device in fluid communication with the chamber for delivering a heated fluid to the chamber to heat the microfluidic device, and a cooling control device in liquid communication with the chamber for delivering a cooled fluid to the chamber to cool the microfluidic device. A temperature control unit in liquid communication with a cooling element and/or a heating element are used to regulate temperature of sequencing substrates and objective lenses for optical detection of sequencing reactions.

This application claims priority to U.S. provisional application No. 61/034,131, filed on Mar. 5, 2008, the entire content of which is hereby incorporated by reference.

### TECHNICAL FIELD

This disclosure relates to temperature control devices for chemical and biological analyses, particularly, in the context of automated DNA sequencing machines and systems.

### BACKGROUND OF THE INVENTION

Traditional nucleic acid sequencing-by-synthesis was commonly performed using various forms of a gel-based method developed by Sanger. Next generation sequencing technologies have sought to move beyond Sanger sequencing and into the realm of more rapid, high-throughput methods at decreased cost. Among these next-generation technologies, are single molecule methods in which individual nucleic acid duplex is observed on a surface and template-dependent base incorporation is recorded for each specific duplex. Single molecule techniques hold promise for rapid sequencing of entire genomes at low cost.

Single molecule technologies generally require sensitive optical detection and the ability to operate at multiple temperatures simultaneously in different parts of the instrument. The present invention solves the problem of thermal control in high-throughput sequencing reactions.

### SUMMARY OF THE INVENTION

The present invention relates to temperature control devices and methods for using them in sequencing reactions. Essentially, the invention provides a temperature control unit in liquid communication with a cooling element and/or a heating element which, in turn are used to regulate temperature of sequencing substrates and objective lenses for optical detection of sequencing reactions.

In a preferred embodiment, the invention provides temperature control for a sequencing apparatus comprising two stages. Each of the two stages comprises a substrate for sequencing that contains a plurality of nucleic acid duplex molecules attached thereto. The duplex comprises a template nucleic acid hybridized to a primer that is extendable at its 3′ end. Sequencing-by-synthesis takes place as follows. The surface is exposed to a polymerase and a nucleotide comprising a detectable label under conditions that allow template-dependent incorporation into the primer. After incorporation, the surface is rinsed to remove unincorporated nucleotides. Then, the surface is ready for imaging of the incorporated nucleotides. This process is repeated multiple times with each of the four nucleotide bases (A, T, C, and G) in order to build a sequence for each template over time as nucleotides are incorporated in each cycle. A detailed description of single molecule sequencing-by-synthesis is found in U.S. Pat. No. 7,282,337, incorporated by reference herein.

In a preferred mode of operation, a sequencing apparatus comprises a plurality of substrates such that sequencing chemistry and imaging can be performed at the same time. In one case, two adjacent substrates are positioned so that chemistry operations are taking place on one, while imaging of incorporated nucleotides is taking place on an adjacent substrate. For single molecule sequencing, it is preferred that the substrates are microfluidic flow cells to which duplex molecules are covalently attached (typically by attachment of the primer portion of the duplex to which the template portion is hybridized). Sequencing chemistry takes place in the microfluidic channels of one of the flow cells and then a stage on which both flow cells are mounted moves to be in proximity of a microscope objective for imaging. An example of a dual flow cell component of a sequencing system is shown in FIG. 1.

In such dual flow-cell formats, it is necessary to conduct sequencing chemistry at a temperature that is higher than the temperature at which optimal imaging is done. This is especially true when fluorescent labels are used to detect incorporated nucleotides. Also, it is important to maintain the microscope objective at an optimal imaging temperature, regardless of the temperature of the flow cells. Accordingly, the invention provides a temperature control apparatus for maintaining appropriate temperature independently in each flow cell and in the objective. The temperature controller, or temperature control apparatus, provides temperature control in the flow cells over a range of temperatures necessary for sequencing and imaging; and is able to switch between temperatures as required when the stage shifts from chemistry to imaging.

A preferred configuration for a temperature controller according to the invention comprises two separate thermal control devices in liquid communication with conduits that carry a liquid to each flow cell, or flow cell mounting chuck, and convey temperature thereto. Separately, the invention contemplates a chiller device to keep the temperature of the microscope objective constant. In one embodiment, the invention comprises a reservoir for storing fluid and a conduit for conveying the fluid to the flow cell or chuck, the reservoir being capable of tunable heating and cooling. The invention contemplates configurations in which each flow cell or chuck is heated/cooled by a separate temperature control module, as well as configurations in which a single control module separately provides temperature control to each flow cell/chuck. Finally, the controller can control temperature to the objective, which typically is the same as the imaging flow cell temperature, or objective temperature control can be done separately.

The optimal temperature for sequencing reactions is about 37° C., however, sequencing can be done at any temperature that is optimal under the desired sequencing protocol. For example, if a melt step is required, the temperature of the “sequencing” flow cell must be raised, preferably to about 70° C. Optimal imaging temperatures are lower than sequencing temperatures, and preferably are about 23° C., but can range between about 17° C. and about 32° C. The temperature of the objective should be the same or about the same as the imaging temperature. For a total internal reflection objective, the optimal temperature is about 23° C.

### BRIEF DESCRIPTION OF THE DRAWINGS

FIG. 1 depicts an example of an apparatus that can be used to perform the processes described below.

FIG. 2 illustrates a dual flow cell assembly with two flow cells.

FIGS. 3A-3C illustrate a flow cell being loaded into one side of a flow chuck. The flow cell 110 is inverted by the user such that the top surface 120 of the flow cell 110 is placed in contact with the flow chuck 490 in the direction indicated by line D in FIG. 3A. As shown in FIG. 3B, the flow cell 110 has the compressible tube 190 disposed in the recess 172 to create a tighter seal when the flow cell 110 is installed in the flow chuck 490. FIG. 3C shows the flow cell 110 mounted in the flow chuck 490 with the top surface 120 of the flow cell in intimate contact with the top surface 494 of the flow chuck 490 and ready for processing by the apparatus 200.

FIG. 4 illustrates a temperature control system according to one exemplary embodiment of the present invention.

### DETAILED DESCRIPTION OF THE INVENTION

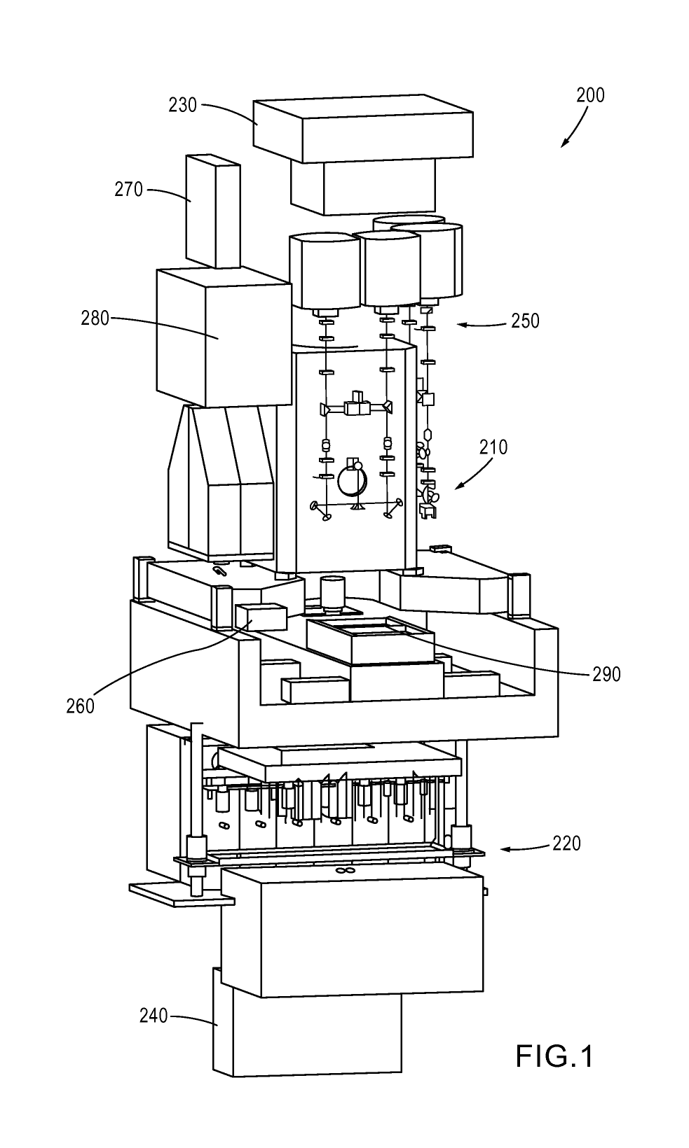

An example of an apparatus 200 that can be used to perform the processes described above is shown in FIG. 1. The apparatus 200 includes an optics section 210, a fluid handling section 220, a filter 230, a power supply 240, a laser control section 250, a bar code reader 260, a motor section 270, a central processing unit 280, and a flow chuck 290. After a flow cell has been prepared for analysis, it may be loaded into the flow chuck 290 of the apparatus 200.

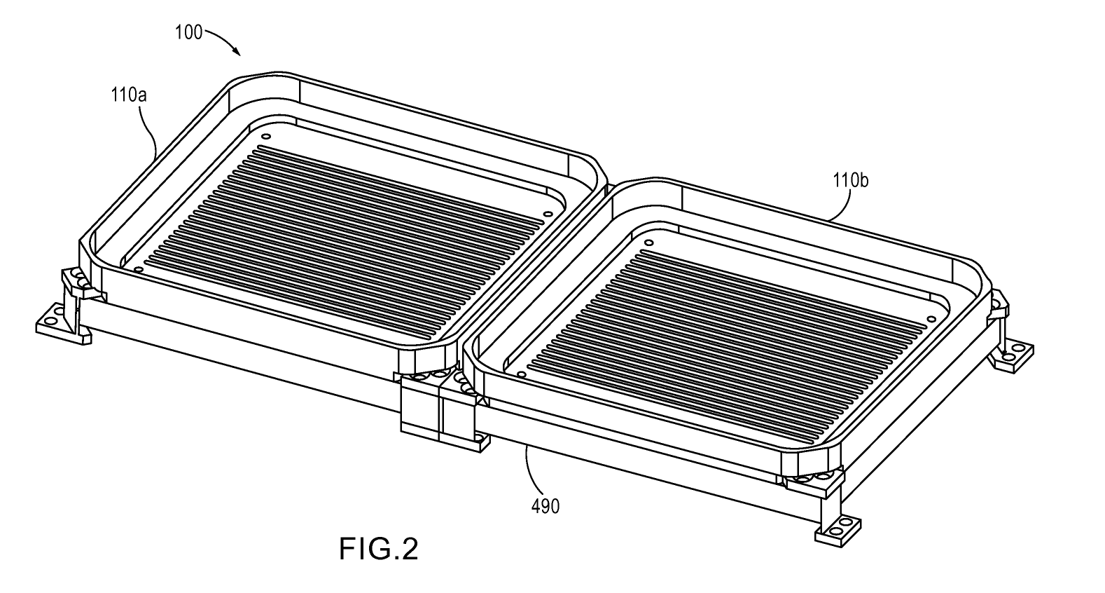

Flow cells can be used individually, or optionally two or more flow cells can be combined together to analyze even more samples simultaneously. As described above, using a dual flow cell assembly allows the apparatus 200 to perform the sequencing chemistry in one flow cell, while at the same time performing the imaging operation in the other flow cell. FIG. 2 illustrates a dual flow cell assembly 100 with two flow cells 110a and 110b (collectively 110) mounted onto a flow chuck 490.

Performing these two operations simultaneously increases throughput of the apparatus 200 by analyzing twice as many samples, but this also requires maintaining several separate components at different temperatures. For example, the optimal temperature for sequencing reactions is about 37° C., however, sequencing can be done at any temperature that is optimal under the desired sequencing protocol. Alternatively, if a melt step is required, the temperature of the “sequencing” flow cell must be raised, preferably to about 70° C. Optimal imaging temperatures are lower than sequencing temperatures, and preferably are about 23° C., but can range between about 17° C. and about 32° C. The temperature of the objective should be the same or about the same as the imaging temperature. For a total internal reflection objective, the optimal temperature is about 23° C.

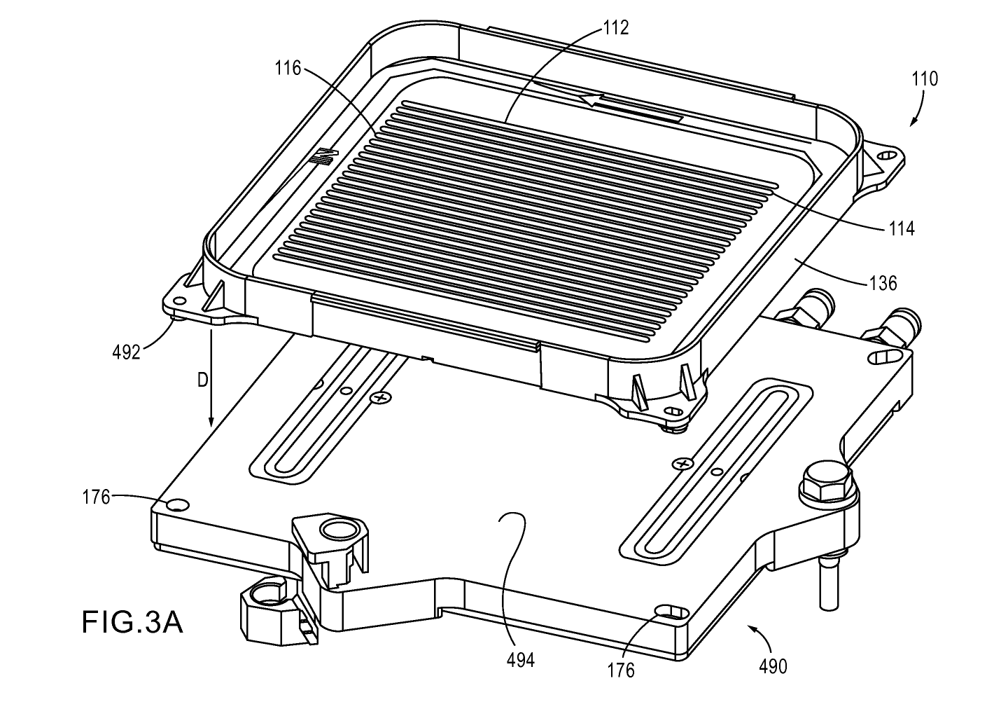

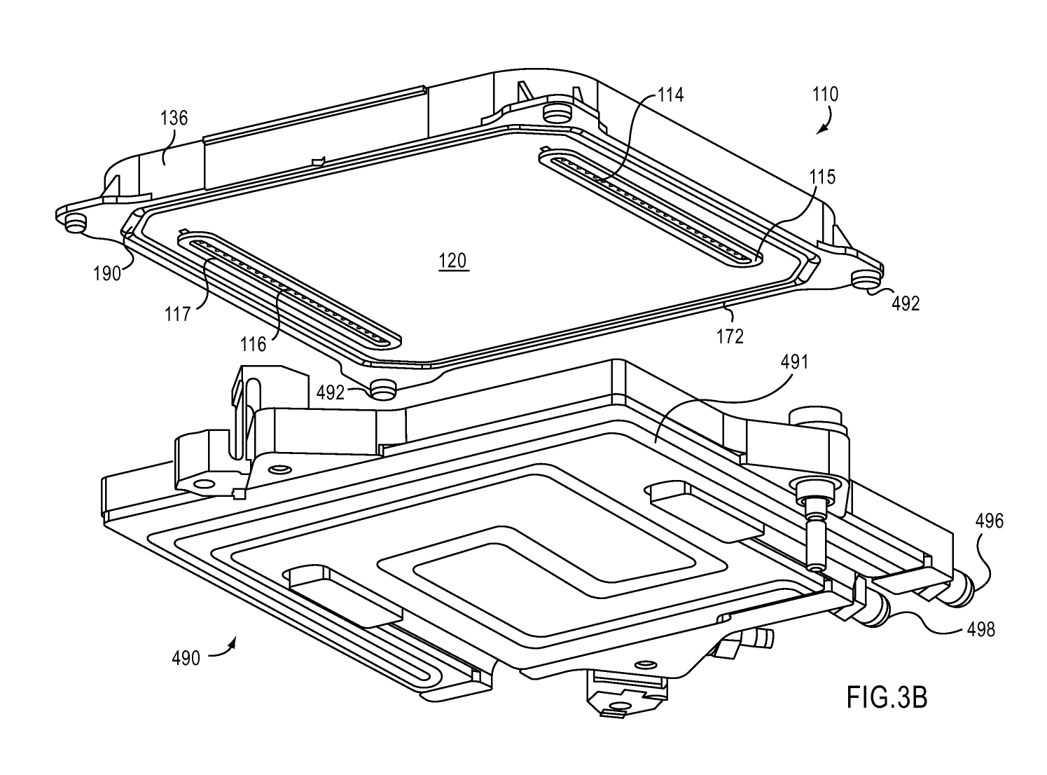

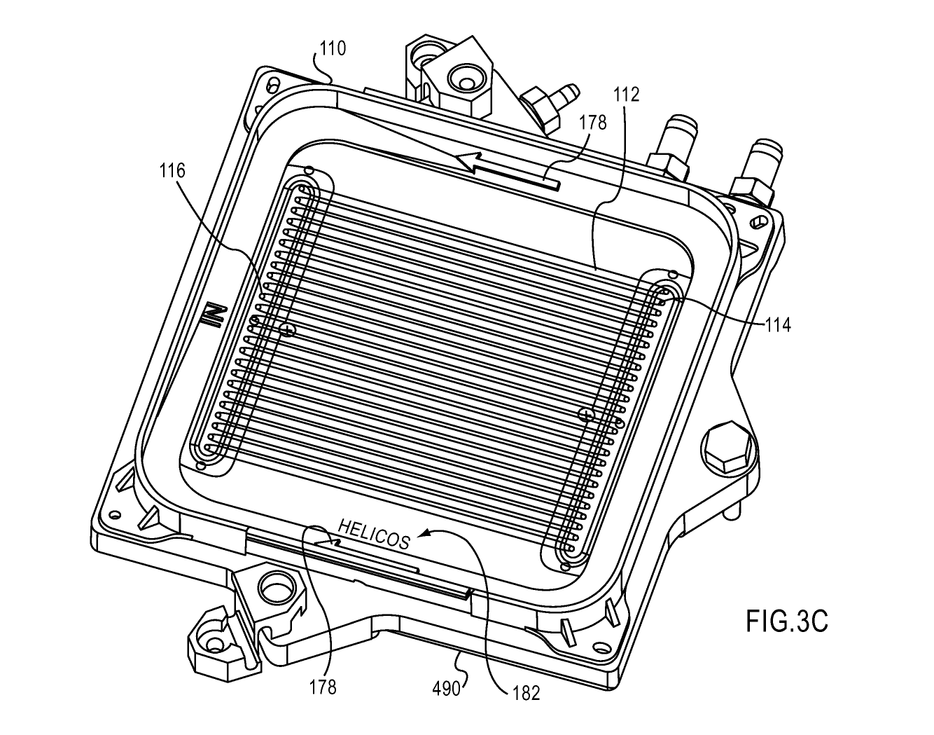

Referring now to FIGS. 3A-3C, a flow cell 110 is being loaded into one side of a flow chuck 490. The flow cell 110 is inverted by the user such that the top surface 120 of the flow cell 110 is placed in contact with the flow chuck 490 in the direction indicated by line D in FIG. 3A. As shown in FIG. 3B, the flow cell 110 has the compressible tube 190 disposed in the recess 172 to create a tighter seal when the flow cell 110 is installed in the flow chuck 490. In this embodiment, the flow cell 110 includes posts 492 and the flow chuck 490 includes slots 176 to ensure proper positioning of the flow cell 110 in the flow chuck 490. The posts 492 also provide protection for the flow cell 110 so that it doesn't break if accidentally dropped or put down improperly on the flow chuck 490. Additional alignment features of this embodiment of the flow cell 110 include arrows 178 and a logo 182. FIG. 3C shows the flow cell 110 mounted in the flow chuck 490 with the top surface 120 of the flow cell in intimate contact with the top surface 494 of the flow chuck 490 and ready for processing by the apparatus 200. Alternate embodiments of the flow cell may also include bar coding or other electromagnetic devices to ensure proper loading and to identify samples that are being analyzed. A second flow cell is loaded into the second side of the flow chuck 490 in the same manner.

Each side of the flow chuck 490 includes an inlet 496 and an outlet 498 fluidly coupled to a chamber 491 (FIG. 3B) such that a heat transfer fluid such as, for example, polyethylene glycol or water, can flow into the inlet 496, circulate though the chamber 491 of the flow chuck 490, and then exit though the outlet 498. The chamber 491 can be a conduit as shown in FIG. 3B or a hollow chamber with baffles to distribute and control the flow thought the chamber 491 and then out through the outlet 498. The specific geometry of the chamber 491 and specific mass flow rates of the heat transfer are designed to provide a uniform temperature profile across the flow chuck 490. The flow chuck 490 is made from a thermally conductive material such as, for example, titanium, aluminum, or stainless steel so heat is readily transferred from the heat transfer fluid to the flow chuck 490 during a heating cycle and from the flow chuck 490 to the heat transfer fluid during a cooling cycle.

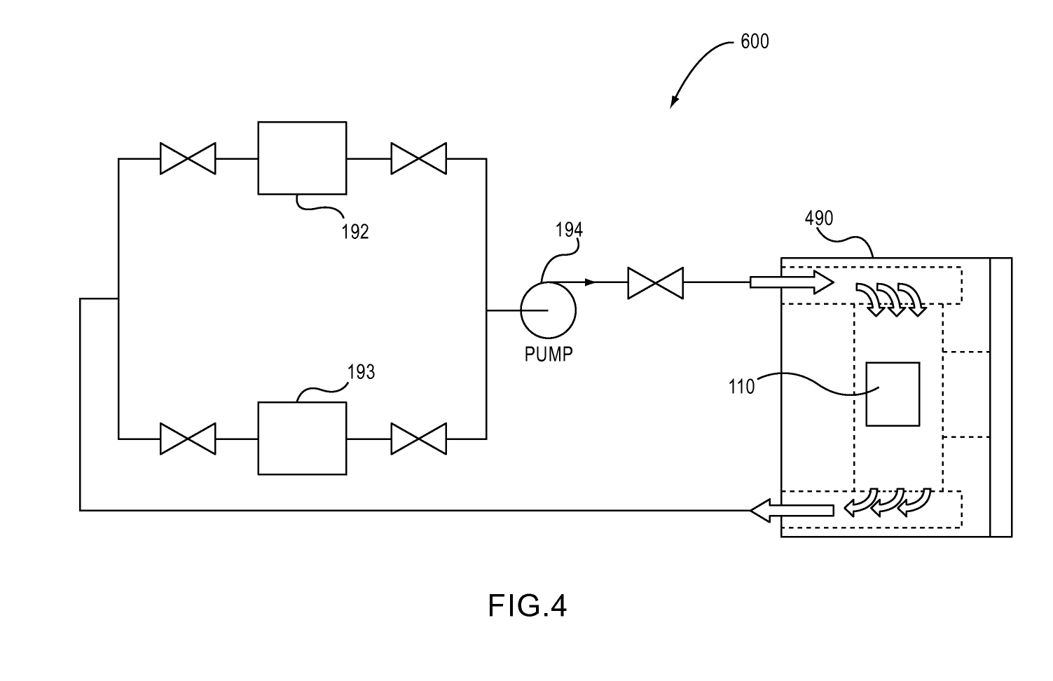

Referring now to FIG. 4, a temperature control system 600 according to one exemplary embodiment of the present invention is shown. The control system includes a heating control device 192 and a cooling control device 193 in liquid communication with the flow chuck 490 via conduits. A flow cell 110 is mounted on a flow chuck 490 as described above. The flow chuck 490 is designed to receive a high heat capacity circulating fluid, such as, for example, ethylene glycol, polyethylene glycol, water, and silicone oil. The flow chuck 490 receives a circulating flow of fluid at a controlled temperature pumped from either the heating control device 192 or a cooling control device 193 by circulating pump 194. A valving arrangement allows for alternating selection between two controlled-temperature storage tanks. Although FIG. 4 shows separate inlet and outlet valves for each tank, equivalent valving arrangements can be used, including valve manifold arrangements and multi-port valves, any of which may be operated manually, pneumatically, or electrically. The temperature of the fluid circulated through the flow chuck 490 is rapidly imparted to the flow cell 110, allowing quick temperature change to be uniformly applied to the flow cell 110.

As shown, the temperature control system 600 is only delivery heated and cools fluid to one side of the flow chuck 490. Additional pumps, valves and conduits can be included to circulate the heated and cooled fluids from the heating control device 192 and the cooling control device 193 to both sides of the flow chuck 490 since both sides need to be maintained at different temperatures. Alternatively two separate temperature control systems 600 can optionally be used.

Although not shown, the cooling control device 193 is also in fluid communication with the optics section to keep the temperature of the microscope objective constant during imaging. The temperature of the microscope objective is typically the same as the imaging flow cell temperature. Alternatively, the objective temperature control can be done separately.

In an alternative exemplary embodiment, the temperature control system 400 comprises a reservoir for storing fluid and a conduit for transporting the fluid to the flow cell 110 or chuck 490, the reservoir being capable of tunable heating and cooling. The invention contemplates configurations in which each flow cell or chuck is heated/cooled by a separate temperature control module, as well as configurations in which a single control module separately provides temperature control to each flow cell/chuck.

In yet another alternative exemplary embodiment, thermoelectric heating and cooling can be used to control the temperature of each of the flow cells and/or the microscope objective. Thermoelectric heating/cooling uses the Peltier effect to create a heat flux between the junction of two different types of materials. A Peltier cooler, heater, or thermoelectric heat pump is a solid-state active heat pump which transfers heat from one side of the device to the other side against the temperature gradient (from cold to hot), with consumption of electrical energy. Such an instrument is also called a Peltier device, Peltier diode, Peltier heat pump, solid state refrigerator, or thermoelectric cooler (TEC). Because heating can be achieved more easily and economically by many other methods, Peltier devices are mostly used for cooling. However, when a single device is to be used for both heating and cooling, a Peltier device may be desirable. Simply connecting the device to a DC voltage will cause one side to cool, while the other side warms. The effectiveness of the pump at moving the heat away from the cold side is totally dependent upon the amount of current provided and how well the heat from the hot side can be removed.

While certain embodiments according to the invention are shown and described, other embodiments are within the scope of this disclosure and are considered to be part hereof. The invention is not to be limited just to certain embodiments shown and/or described.

What is claimed is:

1. A system for controlling the temperature of a microfluidic device comprising:
a chamber for receiving a microfluidic device,
a heating control device in fluid communication with the chamber for delivering a heated fluid to the chamber to heat the microfluidic device; and
a cooling control device in liquid communication with the chamber for delivering a cooled fluid to the chamber to cool the microfluidic device.

2. The system of claim 1, said system adapted for sequencing of nucleic acids.

3. The system of claim 2, wherein said sequencing is sequencing by synthesis.

4. The system of claim 2, wherein said sequencing is single molecule sequencing.

5. A method of conducting nucleic acid sequencing, the method comprising:
a) conducting a template-dependent polymerase-mediated nucleotide addition reaction in a flow cell at 32° C. or higher;
b) lowering the temperature of the flow cell to a detection temperature which is below 32° C.; and
c) optically detecting the result of the reaction in a), thereby determining the identity of one or more incorporated nucleotides;
d) optionally, raising the temperature of the flow cell and repeating steps a)-c).

6. The method of claim 5, wherein the detection in step b) is performed by detecting individual optically resolved molecules.

7. The method of claim 5, wherein the temperature in step a) is approximately 37° C.

8. The method of claim 5, wherein the temperature in step b) is in the range of 17 to 32° C.

9. The method of claim 8, wherein the temperature is in the range of 17 to 27° C.

10. The method of claim 8, wherein the temperature is approximately 23° C.

11. The method of claim 5, wherein step d) is required to be performed at least once or twice.

12. The method of claim 5, wherein step d) is be performed at least 20 times.
